(19) United States
(12) Patent Application Publication
(10) Pub. No.: US 2024/0416136 A1
(43) Pub. Date: Dec. 19, 2024

(54) PLASMA TREATMENT DEVICE FOR PLASMA TREATMENT OF A SKIN SURFACE

(71) Applicant: CINOGY GMBH, Duderstadt (DE)

(72) Inventors: Jan-Hendrik HELLMOLD, Duderstadt (DE); Ronny LETTKE, Duderstadt (DE)

(21) Appl. No.: 18/704,302

(22) PCT Filed: Nov. 2, 2022
(86) PCT No.: PCT/EP2022/080529

§ 371 Date: Apr. 24, 2024

(30) Foreign Application Priority Data
Nov. 2, 2021 (DE) 10 2021 128 469.4

(51) Int. Cl. A61N 1/44

(52) U.S. Cl. CPC A61N 1/44

(57) ABSTRACT

The invention relates to a plasma treatment device for treating a skin surface containing living cells with a dielectric barrier plasma, comprising:
a housing with a grip;
a treatment head arranged on the housing;
an electrode arrangement provided on the treatment head with at least one electrode and a dielectric that completely covers the electrode to the treated surface, said dielectric having a treatment surface and being configured with the at least one electrode in such a way that the at least one electrode engages as a counter electrode with the skin surface to be treated in order to generate the plasma; and
a high-voltage stage arranged in the housing for generating high-voltage signals required for generating the plasma, wherein said high-voltage stage is or can be brought into electrical contact with the at least one electrode of the electrode arrangement by means of a connection arrangement comprising at least one high-voltage supply line;
characterized in that the electrode arrangement is arranged in the housing (11) such that it can be moved and a drive arrangement with at least one motor (30) and one drive train (31) is provided in the housing (11), said drive train being operatively connected with the electrode arrangement for motor-driven movement, while the electrode (19) is in or can be brought into electrical contact with the high-voltage stage (15) via the connection arrangement (35) for the purpose of generating the dielectric barrier plasma between the electrode (19) and the skin surface acting as a counter electrode.

The invention relates to a plasma treatment device for treating a skin surface containing living cells with a dielectric barrier plasma, the skin surface functioning as a counter electrode, according to the preamble of patent claim 1.

It has been known for some time that skin and wound surfaces can be treated advantageously with a dielectric barrier plasma, because the plasma enables reliable disinfection even on areas of the skin surface that are difficult to access, for example, and also prepares the skin surface for the absorption of caring or healing substances and can have a positive effect on wound healing in general, for example by increasing microcirculation in the tissue.

In particular, it has been found that a safe and effective formation of the plasma is possible by using the body belonging to the surface to be treated as a counter electrode (so-called “floating electrode”). This means that the treatment device only needs to have at least one electrode carrying the high voltage and does not require its own counter electrode.

DE 10 2009 060 627 B4 discloses an electrode arrangement composed of a flat flexible electrode and a flexible flat dielectric in which the dielectric surrounds the flat electrode on all sides and only one connection of the electrode is led out of the dielectric in an insulating manner for connecting to a high-voltage generator. The dielectric is intended for contact with the surface to be treated, for example a skin surface of a human or animal body, and has a nub structure on the contact side which acts as a spacer because gas spaces form between the nubs in which the dielectric barrier plasma can be formed.

A treatment device is equipped with a similar electrode arrangement according to DE 10 2012 015 482 A1, wherein the dielectric embedding the electrode forms the front wall of a housing of a treatment device. The flexible electrode arrangement consisting of the flexible dielectric with the flexibly embedded flat electrode is pressed against the surface to be treated by an elastic pressure means arranged behind the electrode arrangement, which improves the adaptability of the electrode arrangement to the contours of the surface to be treated, in particular the skin surface.

DE 10 2013 019 058 A1 and the further developed DE 10 2016 100 466 A1 disclose a treatment device for a dielectric barrier plasma treatment in which the surface to be treated functions as a counter electrode and the dielectric embedding the electrode is formed of a ball protruding from a housing. The ball is rotatably mounted in the housing, the electrode being covered by the dielectric in every possible rotation position. This enables a flexible and free movement of the treatment device across the surface to be treated.

DE 10 2018 126 489 A1 describes a treatment device for a dielectric barrier plasma treatment comprising a brush head as a treatment head that has a plurality of bristles. The gas space formed between the bristles is used to form a dielectric barrier plasma by means of an electrode arrangement.

A treatment device for the skin is known from WO 2017/162505 A1, in which a plasma is generated by means of a first electrode and a second electrode insulated from the first by means of a dielectric, wherein the plasma treatment is to be supported by means of a mechanical manipulator in that areas of the skin that are difficult to access, for example in skin folds, are to be exposed for the plasma treatment. The disadvantage of this is that two electrodes are required in the treatment device to generate a surface plasma, meaning that the device complexity increases while the effectiveness of the plasma treatment decreases.

DE 10 2017 118 568 B3 discloses a plasma treatment device with a treatment head in which the electrode arrangement shielded by a dielectric is located, wherein in the treatment head the electrode arrangement forms a spatially closed flexible sheath around a soft elastic core and is covered on its outer surface by a thin layer of the flexible dielectric, so that the treatment head can assume the shape of the surrounding tissue inside the body when inserted into the body.

DE 10 2015 111 401 B3 describes a treatment device for a dielectric barrier plasma treatment, wherein a treatment head with the electrode and the dielectric comprises a storage chamber for a treatment agent, wherein the volume of said storage chamber can be reduced so that the treatment agent passes through passage openings in the dielectric into the area of the surface to be treated.

DE 10 2019 109 940 A1 describes a treatment device for a dielectric barrier plasma treatment comprising an electrode arrangement and a contacting attachment composed of the dielectric with connecting conductors. The connecting conductors can then by connected to an alternating high voltage source by means of a contacting element, wherein contacting pins of the contacting element contact the connecting conductors through a recess in the dielectric of the contacting attachment.

DE 10 2008 008 034 A1 discloses a polyurethane gel as well as a method for its manufacture.

DE 10 2018 132 918 A1 describes a liner to be applied as padding to an amputation stump, an electrode arrangement for a dielectric barrier plasma discharge being integrated into the liner.

For cosmetic and medical purposes in particular, treatment with a dielectric barrier plasma is also desirable on sensitive areas of skin, such as the face. However, in the cosmetic field in particular it is often necessary to subject the sensitive areas of skin to a mechanical treatment after the plasma treatment in order to achieve the best possible result. This usually requires an additional device, resulting in increased treatment costs.

The present invention therefore aims configure a treatment device of the type mentioned above in such a way that improved treatment, particularly of sensitive skin surfaces, with a dielectric barrier plasma discharge is possible.

According to the invention, the task is solved with the plasma treatment device in accordance with claim 1. Advantageous embodiments of the invention are to be found in the corresponding sub-claims.

According to claim 1, a plasma treatment device in accordance with the preamble is proposed for treating a skin surface containing living cells with a dielectric barrier plasma, the skin surface functioning as a counter electrode. According to the preamble, the plasma treatment device comprises a housing with a grip, a treatment head being arranged on the housing. The treatment head comprises an electrode arrangement that comprises at least one electrode and a dielectric that completely covers the electrode to the skin surface to be treated, the dielectric having a treatment surface which preferably protrudes with a surface section, which includes the treatment surface, out of the treatment head to the surface to be treated. Furthermore, a high-voltage stage is provided in the housing for generating high-voltage signals required for generating the plasma, wherein said high-voltage stage is or can be brought into electrical contact with the at least one electrode of the electrode arrangement by means of a connection arrangement comprising at least one high-voltage supply line. The housing and the grip in particular can be designed to be electrically insulating. The treatment head can be firmly or detachably arranged on the housing. Here, the dielectric is designed in particular to be flexible and is preferably formed from a soft and/or gel-like material.

The high-voltage stage is controlled by a control device in such a way that the high-voltage stage generates the electrical high-voltage signals required to generate the dielectric barrier plasma and emits them to the at least one electrode. The high-voltage stage can be designed in such a way that the high-voltage signals are generated as individual pulse signals in the form of individual pulses, in the form of pulse trains and/or in the form of damped oscillations and emitted to the electrode. In the case of a pulse train with damped oscillation, the start wave has the highest amplitude. The high-voltage stage is therefore a high-voltage generator, which transforms an input voltage into a high voltage in the manner of a high-voltage transformer.

According to the invention, it is now provided that the electrode arrangement is arranged in the housing such that it can be moved and a drive arrangement with at least one motor and one drive train is provided in the housing, said drive train being operatively connected with the electrode arrangement for motor-driven movement, while the electrode is in electrical contact with the high-voltage stage via the connection arrangement for the purpose of generating the dielectric barrier plasma between the electrode and the skin surface acting as a counter electrode. In particular, the electrode arrangement is operatively connected to a part of the drive train so that the movement of the motor is transferred to the electrode arrangement.

The plasma treatment device is therefore designed to move the electrode arrangement during the generation of the dielectric barrier plasma between the electrode and the skin surface functioning as a counter electrode in order to support the treatment by way of a mechanical movement of the electrode arrangement while the dielectric barrier plasma is being generated. To this end, the treatment surface of the dielectric is contacted with the skin surface to be treated during the treatment, so that the dielectric barrier plasma forms between the treatment surface and the skin surface and at the same time, the plasma treatment is supported by a movement due to the friction between the treatment surface of the dielectric and the skin surface. The high-voltage signals required to generate the plasma are safely transmitted from the high-voltage stage from the stationary system of the housing to the moving system of the electrode arrangement with the aid of the electrical connection arrangement in order to generate the dielectric barrier plasma. The movement of the electrode arrangement can be a rotating, rotationally oscillating and/or a translational movement, e.g. in the form of a vibration, as well as combinations thereof. A translational movement, for example, may be a to-and-fro movement in which, similarly to the rotationally oscillating movement, the movement direction of the translational movement changes periodically. In particular, the change in movement direction may be in the opposite movement direction.

A rotating movement of the electrode arrangement is understood to mean a rotation of the electrode arrangement about a rotational axis, wherein the direction of rotation does not change. A rotationally oscillating movement of the electrode arrangement is understood to mean a rotating movement of the electrode arrangement in which the direction of rotation changes periodically. Preferably, the angle of rotation for each direction of rotation is smaller than a full circle, preferably smaller than a semi-circle and particularly preferably smaller than a quarter circle.

It can be provided for that the treatment surface of the dielectric is designed to be largely flat. This is to be understood particularly to mean that the dielectric is not spherical. In the context of the present invention, a flat form of the dielectric at the treatment surface is understood to mean that the plane of the treatment surface is largely level or flat. However, a largely flat treatment surface is understood to be a still acceptable curvature (predetermined or due to manufacturing tolerances), whereby the degree of curvature (ratio of centre angle to length of the circular arc viewed in a spatial direction) is less than 50%. The dielectric may, however, also be semi-spherical.

The electrode arrangement can be operatively connected to part of the drive train by means of a form fit so that, with the aid of the form fit, the movement of the motor is transmitted to the electrode unit.

According to one embodiment, it is provided that the electrode arrangement is detachably arranged or insertable on the housing, wherein the connection arrangement connects the at least one electrode to the high-voltage stage when the electrode arrangement is movably mounted on the housing in an inserted position. In this case, the electrode arrangement can be brought from a detached position into an inserted position and vice-versa. In the detached position, the electrode arrangement (or possibly the entire treatment head) is removed from the housing or is not operatively connected to the drive train, whereas in the inserted position, the drive train is inserted into the housing and is operatively connected to the drive train of the drive arrangement in the housing in such a way that the electrode arrangement can be moved.

According to one embodiment, it is provided that the electrode arrangement is detachably arranged or insertable on the housing by means of a snap-on connection. With the aid of the snap-on connection, the electrode is fixed axially (e.g. to a rotational axis) so that the electrode arrangement cannot be moved in the axial direction. The axial fixing allows for a desired movement of the electrode arrangement during the plasma treatment, e.g. a movement about a rotational axis for a rotating or rotationally oscillating movement of the electrode arrangement. The detachment of the snap-on connection can be achieved by overcoming a corresponding connection force or by actuating an ejector mechanism.

For example, such a snap-on connection can be achieved by providing at least one spring-loaded tappet in the housing which engages via a snap-on section in a recess radial to a rotary shaft or drive shaft of the electrode arrangement when the electrode arrangement is properly mounted in the housing. The at least one spring-loaded tappet applies a spring force in the direction of the rotary shaft or drive shaft of the electrode arrangement (essentially perpendicular to it) when the electrode arrangement is mounted on the housing in the inserted position and engages via its snap-in section in a recess within the rotary shaft or drive shaft of the electrode arrangement so as to prevent an unwanted movement of the electrode arrangement transverse to the tappet (i.e. in the axial direction). In this case, the recess provided in the rotary shaft or drive shaft of the electrode arrangement may be circumferential (quasi infinite) so that the electrode arrangement can still be rotated about the rotational axis. Other snap-in connections are conceivable, however, which ensure temporary securing of the treatment head (in particular axially to the rotational direction). For example, it is conceivable that spring-loaded balls are provided in the rotary shaft or drive shaft that protrude with a particular ball section from the rotary shaft or drive train and are spring-loaded radially to the rotary shaft or drive train. Said spring-loaded balls can now engage in a recess within the guide channel of the housing, into which the electrode arrangement is inserted, and thus fix the electrode arrangement axially to the rotational direction.

According to a preferred embodiment, it is provided that the plasma treatment device has at least one positioning sensor (measuring sensor) that is designed to detect the inserted position of the electrode arrangement in the housing, wherein the high-voltage stage is switched off unless the inserted position is detected by means of the at least one position sensor. Accordingly, the inserted position of the electrode arrangement and possibly of the treatment head in the housing is detected by means of the position sensor, wherein the plasma treatment device is further configured to switch the high-voltage stage into an activated operating state when the inserted position is detected by means of the at least one position sensor. The high-voltage stage otherwise remains switched off.

This ensures that the high-voltage stage can only be activated when the electrode arrangement and possibly the treatment head is inserted in the housing in the inserted position, whereby an unintentional spark-over from the counter contact provided in the housing can be avoided. A treatment device that is mistakenly put into operation without the electrode arrangement in place will therefore not generate the high-voltage signals required to generate the plasma that would act on the counter contact. Only when the inserted position is detected by the position sensor is it ensured that the counter contact in the housing is in contact with the electrode and a spark is prevented due to the dielectric.

Such a position sensor or measuring sensor can be realized, for example, with the aid of a position switch. In the inserted position, the position switch is held in an on position (NO switch, “normally open”), which establishes a voltage supply with the high-voltage stage and/or the control device. In the released position, in which the electrode arrangement is not mounted in the housing, the position switch is in an off position, whereby the voltage supply for the high-voltage stage and/or the control device is uninterrupted. An accidental actuation of the treatment device does not therefore lead to the generation of high-voltage signals.

Alternatively or additionally, the inserted position of the electrode arrangement and possibly the treatment head can also be detected with the aid of a continuity tester. To this end, at least two connected conductive contacts are provided, for example, on the electrode arrangement or the treatment head, which are each in electrical contact with a continuity tester in the inserted position. On the basis of a possible current flow between the two conductive contacts, said continuity tester can now determine whether the electrode arrangement or the treatment head is correctly mounted in the inserted position. An accidental actuation of the treatment device does not lead to the generation of high-voltage signals in this case either as long as the continuity test is negative.

Other options for determining whether the electrode arrangement is correctly connected to the housing include the detection of an RFID chip connected to the electrode arrangement, the use of an inductive or magnetic measuring sensor or similar.

According to a preferred embodiment, it is provided that the at least one electrode is led out of the dielectric with an electrical connecting bolt on a rear side of the electrode arrangement opposite the treatment surface, wherein the electrical connecting bolt has a contact surface on an end section opposite the electrode, which engages with a counter contact that is provided in the housing and is electrically connected to the high-voltage stage for electrical contacting when the electrode arrangement is movably mounted on the housing in the inserted position. Such a connecting bolt may be the rotary shaft or drive shaft that is operatively connected to the drive train of the motor arrangement for the purposes of movement. In other words, the connecting bolt is operatively connected to the drive train of the drive arrangement for the purpose of motor-driven movement when the electrode arrangement is mounted on the housing in the inserted position. As a result, the connecting bolt not only forms part of the connection arrangement in order to feed the electrode, which is in electrical contact with the connecting bolt, with high-voltage signals to generate the dielectric barrier plasma, but it also forms part of the drive train to move the electrode arrangement and possibly the treatment head with the aid of the motor, e.g. in a rotating or rotationally oscillating manner.

The contact surface to the electrical contacting with the counter contact provided in the housing can be the front side or face of the connecting bolt. Here, a spring-loaded counter contact may be arranged in the housing that presses on the front contact surface and simultaneously enables a rotating or rotationally oscillating movement. The contact surface may however also be the circumferential surface or lateral surface of the end section of the connecting bolt, wherein in this case, the counter contact is pressed against the circumferential surface or lateral surface. For example, such a counter contact may be a spring clamping shoe.

According to one embodiment, it is provided that the connecting bolt forms a form fit and/or force fit with at least part of the drive train that acts about the longitudinal axis of the connecting bolt when the electrode arrangement is mounted on the housing in the inserted position, particularly in a rotating or rotationally oscillating manner.

A form fit can be easily achieved in that the connecting bolt has a cross-section that corresponds to a cross-section of a drive train mount in the housing so that the connecting bolt can be inserted with its particular cross-section into the drive train mount, both thus forming the form fit by means of which a movement of the drive train is transmitted to the connecting bolt and thus to the electrode arrangement. For example, such a cross-section may be a polygon (larger/equal to three corners), such as a hexagon.

According to one embodiment, it is provided that the electrode arrangement comprises a flat carrier to which the dielectric is fixed to a dielectric support and from which the connecting bolt is led out on the rear side of the carrier opposite the dielectric. The dielectric is fixed to the dielectric support on the flat carrier (detachably or permanently). In this embodiment, therefore, the treatment head is formed of the carrier as well as the electrode arrangement with the dielectric and electrode as well as a connection arrangement for connecting the electrode with the high-voltage stage.

According to one embodiment, it is provided that the flat carrier of the treatment head comprises a recess around the connecting bolt on the rear side, with which the flat carrier is connected in a torque-proof manner to at least one part of the drive train when the electrode arrangement is mounted on the housing in the inserted position, especially in a rotating or rotationally oscillating manner.

In this embodiment, the recess can comprise a cross-section that corresponds to the cross-section of the part of the drive train which engages in the recess in the carrier, thus forming the form fit between the drive train and the carrier. In this case, the function of the electric contacting can be decoupled from the mechanical connection for transmitting the rotating or rotationally oscillating movement in particular.

According to one embodiment, it is provided that the connecting bolt is at least partially surrounded by an electrically insulating cover.

The insulating cover with the led-out end of the electrode (electrical connecting bolt) can be inserted into a mount in the housing of the plasma treatment device and detachably held in the housing by means of a snap-on connection. As soon as the electrode arrangement has been brought into the inserted position, the snap-in connection engages in specially provided elements, thus securely holding the electrode arrangement and, where applicable, the treatment head in the inserted position.

According to one embodiment, it is provided that the electrically insulating cover protrudes beyond the contact surface of the electrical connecting bolt, so that electrical contact is made with the counter contact inside the insulating cover when the electrode arrangement is mounted on the housing in the inserted position.

The counter contact may be a spring contact in the housing which contacts the contact surface of the electrical connecting bolt when the electrode arrangement is inserted into the inserted position and presses against said contact surface with a certain spring force so as to ensure secure electrical contacting. In this case, it can be provided that the counter contact contacts the contact surface for a high-voltage contacting (HV contacting) within the insulating cover in order to ensure the highest possible operating safety and prevent the risk of a spark.

However, it is also conceivable that the contact surface of the electrical connecting bolt protrudes beyond the electrically insulating cover, so that the electrical contacting with the counter contact occurs outside of the insulating cover in a mount in the housing when the electrode arrangement is mounted on the housing in the inserted position. This is particularly advantageous if the counter contact is made on the lateral surface in the end section of the connecting bolt.

According to one embodiment, it is provided that the dielectric of the electrode arrangement is formed at least partially from a gel-like material, in particular on a polyurethane basis. In particular, it can be provided that the dielectric of the electrode arrangement is formed at least partially from a polyurethane gel, which is not a hydrogel.

Surprisingly, such a polyurethane gel is suitable as a dielectric, so that a very soft and highly flexible dielectric can be provided on a plasma treatment device for generating a dielectric barrier plasma, which is particularly suitable for treating sensitive skin areas. The gel-like dielectric is preferably equipped with at least a soft and tear-resistant skin on the dielectric surface, so that contact with the dielectric can be used to treat sensitive skin areas and there is no risk of accidentally touching the electrode embedded in the dielectric. This is because the tear-resistant dielectric surface, which is created by forming a skin of the polyurethane gel or preferably by applying a film, which may be made of silicone or preferably of polyurethane, to the gel during a casting process to shape the dielectric, prevents mechanical destruction of the treatment surface of the dielectric, which would create the risk of a spark from the electrode to the person concerned, and at the same time creates a pleasant and gentle treatment of sensitive skin areas. The soft and supple polyurethane gel is produced from the reaction of at least one polyol with at least one suitable isocyanate, wherein the immobilized disperse (liquid) phase of the gel is formed by the at least one polyol and not—as in the case of a hydrogel—by water. Rather, the water content of the polyurethane gel is less than 5% by weight, preferably less than 3% by weight, due to the absorption of moisture after manufacture. The skin, which can completely enclose the polyurethane gel, has a thickness of less than 1 mm, preferably less than 0.5 mm, particularly preferably less than 0.2 mm, and is so flexible that the advantageous mechanical properties of the gel (softness, adaptability to curved surfaces, pleasant contact feel) are not impaired.

It is therefore possible to overcome the disadvantage of known plasma treatment devices for generating a dielectric barrier plasma when treating sensitive skin areas.

It was recognized that a polyurethane gel, which is not a hydrogel, can be formed as a dielectric of an electrode arrangement for generating a dielectric barrier plasma and can be used for this purpose.

The dielectric can be formed entirely from the polyurethane gel and is designed to be electrically insulating. However, it is also conceivable that the dielectric is composed of at least two layers and that only the first layer of the dielectric comprising the treatment surface is formed from the polyurethane gel, while the remaining layers of the dielectric are made of another flexible material, such as silicone. The electrode can be completely embedded between the layers or exclusively in the first or preferably in the second layer.

The polyurethane gel may be a sol-gel, for example. Such a gel-like material is produced in a sol-gel process. In particular, the gel can be made and formed without plasticizers and has the dimensional stability of a solid with elastic properties. Its electric properties allow it to be used in accordance with the invention as a dielectric for generating a dielectric barrier plasma.

In particular, the gel-like material may be a material that is pourable during the manufacturing process, wherein the dielectric is manufactured in a mold by pouring in the gel-like material. It is conceivable that a suitable film is inserted into the mold to form the skin, with the gel-like material then being applied to said film by pouring it into the mold. The embedded electrode can be manufactured with the dielectric in that the electrode is already in the desired position within the mold and is surrounded by the pourable material during filling. However, it is also conceivable that the dielectric is made up of two or more layers produced individually or separately, the at least one electrode then being embedded between two layers. However, it is also conceivable that the dielectric is manufactured as a single piece, but is composed of multiple layers of different materials that are brought together during the manufacturing process and form a material bond at the boundary layer.

The at least one electrode is preferably completely surrounded by the dielectric material (e.g. gel) and embedded in said material as a dielectric.

The dielectric can, however, also be made of conventional materials, such as silicone.

According to one embodiment, it is provided that the treatment surface of the dielectric has a spacer with elevations and depressions between them for forming a gas space. Such a spacer can be formed with the aid of a plurality of nubs, between which there are depressions that form the gas space required to generate the plasma. Another embodiment provides a lattice-like wall structure as a spacer, the walls preferably being thin with a thickness that is small compared to the dimensions of the air spaces that they form.

According to a preferred embodiment, it is provided that an electric energy store is provided in the housing that is configured to supply the high-voltage stage with electrical energy. The electric energy store may be a battery. The electric energy store may, however, also be rechargeable in the form of a rechargeable battery. This makes it possible to operate the plasma treatment device autonomously without an external energy source for a certain period of time, whereby it was determined that with the aid of such a mobile energy store in the manually operated treatment device, a corresponding dielectric barrier plasma discharge can be operated over a certain period of time, in particular together with a driven moving electrode arrangement.

Alternatively or additionally, it can be provided that the high-voltage stage is or can be connected to an external energy source. This makes it possible to charge an energy store in the treatment device by way of the external energy source. However, it is also conceivable that the external energy source is configured to directly supply the high-voltage stage with electrical energy, such that a mobile energy store does not need to be deployed in the device.

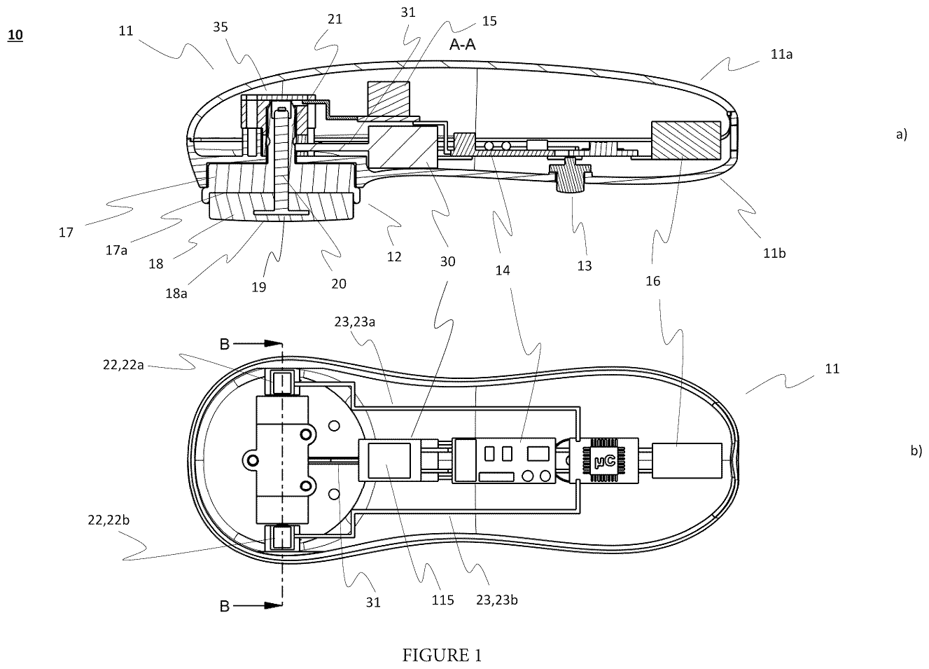

FIG. 1 sectional view of the plasma treatment device according to the invention in one embodiment;

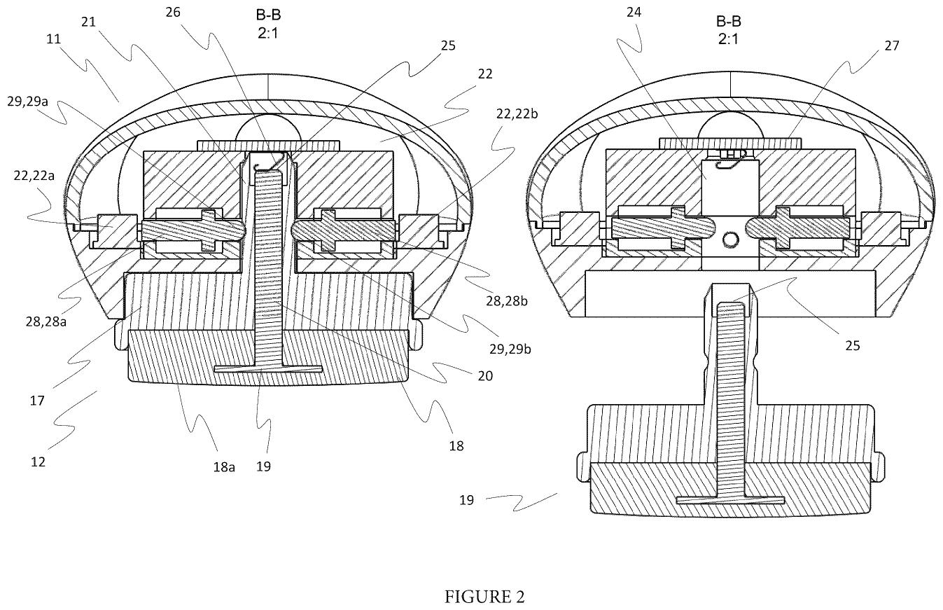

FIG. 2 sectional view through the treatment head of the plasma treatment device from FIG. 1;

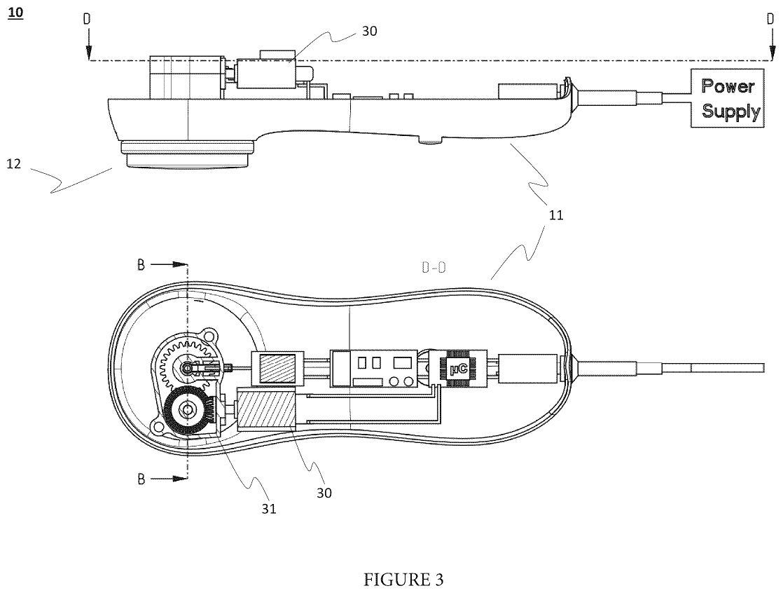

FIG. 3 plasma treatment device in a detailed illustration of the drive arrangement;

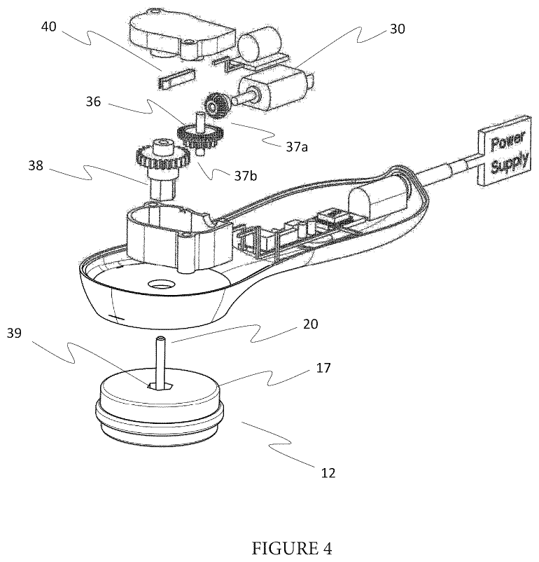

FIG. 4 exploded view of the plasma treatment device from FIG. 3;

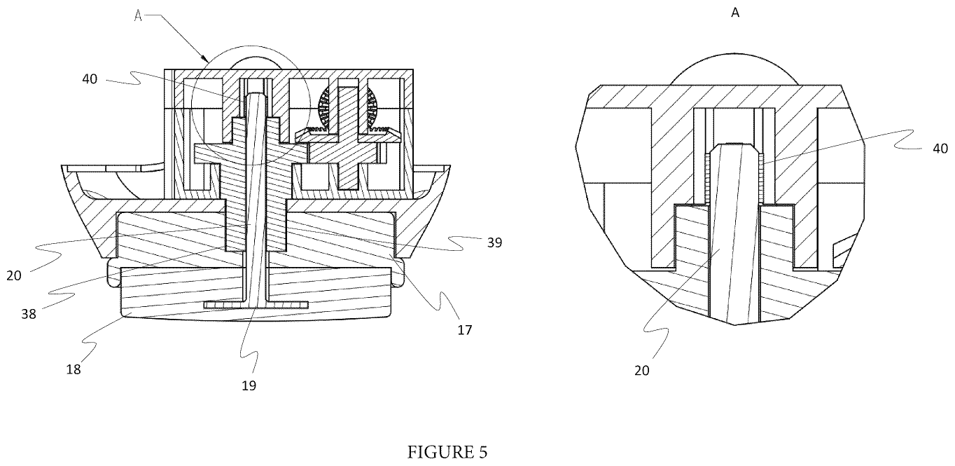

FIG. 5 sectional view of a treatment head from FIGS. 3 and 4;

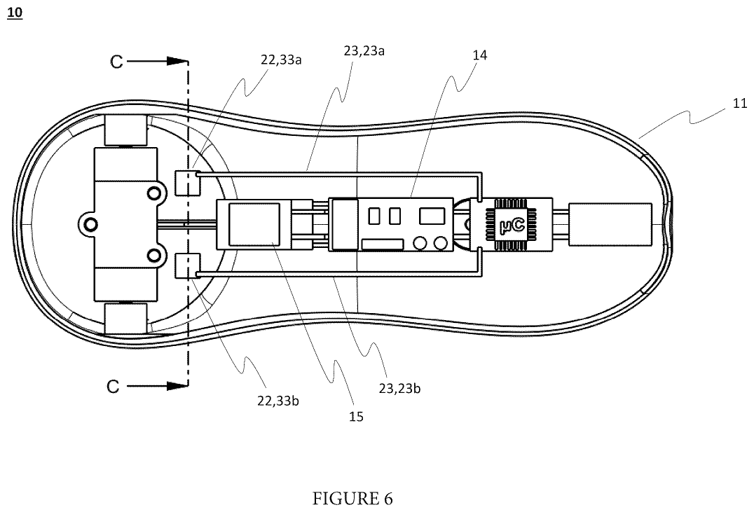

FIG. 6 sectional view of a plasma treatment device in one embodiment with a continuity tester;

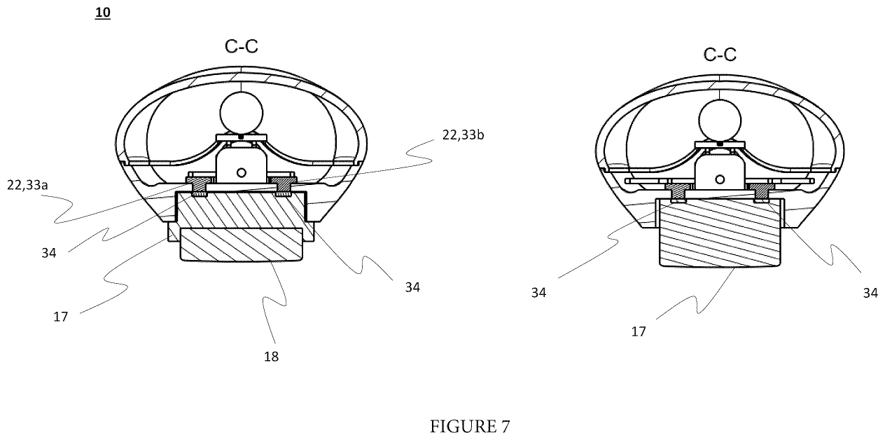

FIG. 7 sectional view through the treatment head of the plasma treatment device from FIG. 6 in two alternative embodiments;

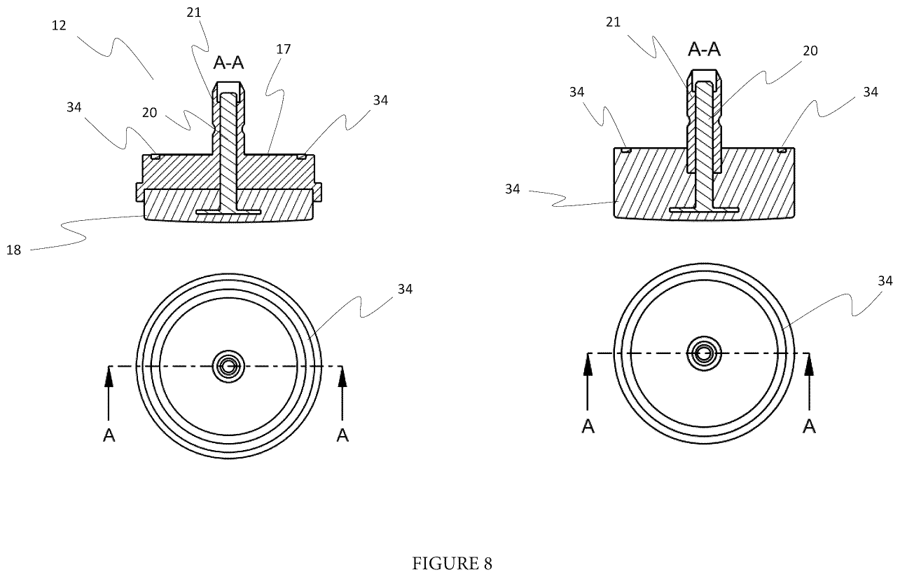

FIG. 8 sectional view through the electrode arrangement of the two alternative embodiments from FIG. 7;

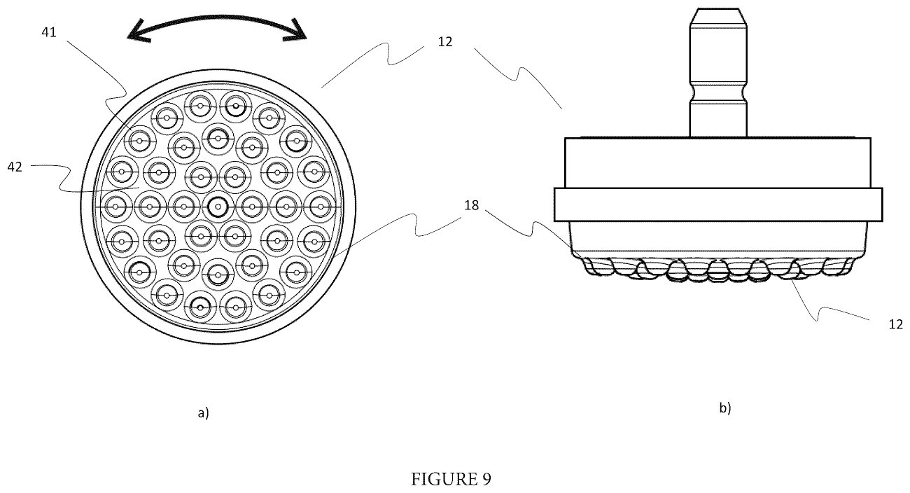

FIG. 9 representation of a treatment head in a further embodiment;

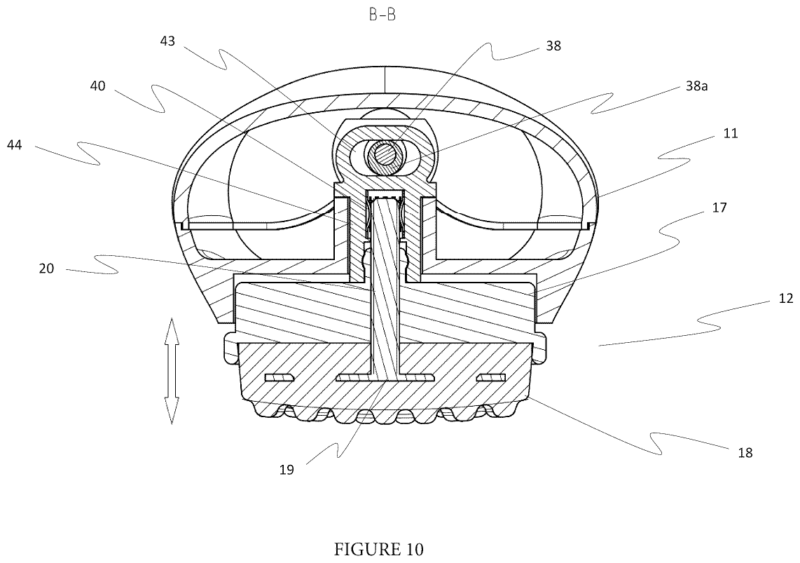

FIG. 10 representation of an embodiment with translational movement, the movement direction of which changes periodically.

In FIG. 1, the upper illustration a) shows a lateral cross-section, while the lower illustration b) shows a top view as a sectional view. A plasma treatment device 10 can be recognized, which has a housing 11 and a treatment head 12. In the embodiment example from FIG. 1, the housing 11 has an upper housing part 11a and a lower housing part 11b, wherein the treatment head 12 is being inserted into the housing 11 in the lower housing part 11b. An actuation button 13 is also arranged in the lower housing part 11b for activating and/or deactivating the treatment device 10.

A control device 14 is accommodated in the housing 11, the former being operatively connected to a high-voltage stage 15. The high-voltage stage 15 in the form of an ignition coil or a transformer is configured to generate high-voltage signals in order to generate the plasma. Actuating the actuation button 13 activates the control device 14 which, with the aid of the high-voltage stage 15, triggers the generation of the high-voltage signals for generating the dielectric barrier plasma. Furthermore, another actuation button may be provided, with which the movement of the electrode arrangement can be started independently from the plasma.

The control device 14 is connected to an electric energy source 16 in the form of a rechargeable battery, which provides the electrical energy required to operate the control device 14 and to generate the high-voltage signals. The rechargeable battery can be led out with an interface on the outside of the housing in order to charge the rechargeable battery. However, it is also conceivable that the rechargeable battery is charged inductively with the aid of an inductive charging device.

In the embodiment example from FIG. 1, the treatment head 12 is in the inserted position and also comprises a flat carrier 17, on which a dielectric 18 is arranged which, together with a flat electrode 19, forms an electrode arrangement. The dielectric 18 can be attached to the dielectric support 17a of the carrier 17 in a non-detachable manner, for example by gluing.

The electrode 19 is embedded in the dielectric 18, the electrode being led out of the dielectric 18 by means of a connecting bolt 20 and protruding through the carrier 17 far into the housing 11. An extension projecting away from the rear of the carrier 17 forms an electrically insulating cover 21, which is part of the connection arrangement 35 for fastening the treatment head 12 to the housing 11, in particular in a detachable manner.

The dielectric 18 of the electrode arrangement is made of an electrically insulating, gel-like polyurethane-based material and has a treatment surface 18a, which is placed on the skin surface to be treated. The treatment surface 18a can have spacers (FIG. 9)—preferably molded into the dielectric—which contain depressions so that a gas space is formed for the formation of the dielectric barrier plasma. Such a spacer can be formed from a plurality of nubs or thin grid-like walls.

By means of the connection arrangement 35, the electrode 19 of the treatment head 12 is electrically contacted with the high-voltage stage 15 when the electrode arrangement is mounted in the inserted position in the housing 11, so that a high-voltage signal generated by the high-voltage stage 15 can be transmitted to the electrode 19 for generating a dielectric barrier plasma.

In order to avoid the risk of an accidental spark-over when actuating the actuation button 13 when the treatment head 12 is not inserted in the housing 11, position sensors 22a, 22b (measuring sensors) are provided, which are connected to the control device 14 via electrical lines 23. If correct insertion of the treatment head 12 is detected with the aid of the positioning sensors 22, the circuit of the respective line 23 is closed, causing the control device 14 to be supplied accordingly with electrical energy.

The electrode arrangement is rotatably mounted in the housing 11 such that it can be rotated about a rotational axis. The rotational axis is designed to be axial or coaxial to the connecting bolt 20. Furthermore, a motor 30 is provided in the device 10, said motor being connected to the control device 10 and driving the electrode arrangement via a drive train 31 in a rotating or rotationally oscillating manner. The motor 30 and drive train 31 form a drive arrangement. To this end, for example, a sprocket can be provided on the circumference of the electrically insulating cover 21, which engages in a corresponding gear wheel of the drive train 31 and thus drives the electrode arrangement and possibly the treatment head 12 in a rotating or rotationally oscillating manner.

On the left-hand side of FIG. 2 is a sectional view B-B (see FIG. 1) with the electrode arrangement in the inserted position; on the right-hand side is a sectional view with the electrode arrangement in the released, non-inserted position, so as to show the snap-in connection in detail. The connection arrangement 25 accommodated in the housing 11 has a guide channel 24 into which the extension of the carrier 17, which forms the electrically insulating cover 21 of the connecting bolt 20 of the electrode 19, can be precisely inserted. The electrically insulating cover 21 is designed to be longer than the connecting bolt 20 at the end opposite the electrode 19 so that the contact surface 25 lies within the electrically insulating cover 21.

The connecting arrangement 35 also has a spring contact 26, which acts as a counter contact to contact the contact surface 25 of the connecting bolt 20 when the treatment head 12 is mounted in the inserted position in the housing (left-hand illustration). Conversely, in the non-inserted position (right-hand illustration) there is no electrical connection between the spring contact 26 and the contact surface 25. Since the electrically insulating cover 21 protrudes beyond the end of the contact surface 25 of the connecting bolt 20, electrical contacting with the spring contact 26 occurs inside the electrically insulating cover 21.

The spring contact 26 can be arranged on a circuit board 27 for contacting, the circuit board 27 having electrical contact means for electrically connecting the high-voltage stage 15 and/or the control device 14 to the spring contact 26.

The connecting arrangement 35 has a snap-in connection which has a total of two spring-loaded tappets 28 in the embodiment example in FIG. 2. The tappets 28 are mounted in such a way that they can apply a radial spring force in the direction of the electrically insulating cover 21. At the end of the tappets 28 facing the electrically insulating cover 21 is a snap-in section 29, which engages or snaps into a (snap-in) depression in the electrically insulating cover 21 when the treatment head 12 is inserted, thereby holding the treatment head 12 in the inserted position. The depression in the electrically insulating cover 21 is designed to be circumferential so that the electrode arrangement is rotatably mounted, wherein the rotational axis lies within the connecting bolt 20. The spring contact 26 ensures constant contacting of the electrode 19, even if the electrode arrangement and possibly the treatment head 12 rotates about the rotational axis.

In the inserted position, the tappets 29 are reset and thereby actuate the position sensor 22 on the respective side, whereby the position sensor 22 closes the circuit from FIG. 1 and thus sets the electronics in the device to an activated operating state.

If the electrode arrangement is removed along with the treatment head 12 from the housing from the inserted position, as shown on the right-hand side of FIG. 2, the tappets 29 are pressed into the guide channel 24 by the spring-loaded mounting up to an end stop, wherein in this position the position sensor 22 is not actuated and the circuit is therefore open, whereby the electronics in the device are set to a deactivated operating state. It is inevitably within the specialist's reach that, in addition to an NC sensor (NC=normally closed), NO sensors (NO=normally open) can also be used, as well as a push-button as a position sensor 22.

FIG. 3 shows an embodiment example in which the plasma treatment device 10 is connected to an external energy source 32, so that a rechargeable battery or a battery inside the housing is rendered unnecessary. The external electrical energy source 32 is tapped via a cable.

Moreover, a further embodiment of the drive arrangement is depicted in FIG. 3 in connection with FIG. 4 (exploded view) and in FIG. 5 in detail, said arrangement serving to move the treatment head 12 in a rotating or rotationally oscillating manner. The motor 30, which is accommodated in the housing 11, is detachably connected to the electrode arrangement via a drive train 31, in particular in order to transmit a rotating movement initiated by the motor 30 to the drive train 31 and then to the electrode arrangement that is operatively connected to the drive train 31.

The drive train 31 can be composed of multiple gearwheels 60, by means of which a certain translation ratio for the rotational speed of the electrode arrangement is stipulated. Since the motor, with its rotational axis, lies longitudinal in the housing, the angle of rotation is first of all altered by a first gearwheel arrangement 37a; the rotational movement is subsequently transmitted to the electrode arrangement by a second gearwheel arrangement 37b (see FIG. 4).

In the embodiment example in FIGS. 3 to 5, the second gearwheel arrangement 37b comprises a drive shaft 38, which comprises a hexagonal cross-section at its end facing towards the treatment head 12. The cross-section of the drive shaft 38 form-fits into a recess 39 in the carrier 17 of the treatment head 12 when the treatment head 12 is mounted in the housing in the inserted position (see FIG. 5).

The recess 39 is provided around the connecting bolt 20 of the treatment head 12 in the carrier 17, so that the connecting bolt 20 forms the rotational axis of the treatment head 12. In the inserted state, the drive shaft 38 therefore engages with its fitting end in this recess 39 in the carrier 17 of the treatment head 12 and thus forms a rotary form-fit, so that rotational movements of the drive shaft 38 are transmitted to the treatment head 12. As a result, the motor 30 can transmit its rotational movement to the electrode arrangement in the treatment head 12.

In the embodiment example in FIGS. 3 to 5, the drive shaft 38 is designed to be hollow, so that the connecting bolt 20 can be inserted into it and protrudes by a certain amount at the opposite end of the drive shaft 38 (see FIG. 5). In this case, the connecting bolt 20 is not depicted with an electrically insulating cover; it may however have one under certain circumstances. The end of the connecting bolt 20 that protrudes from the drive shaft 38 at the opposite end of the treatment head 12 contacts a clamp spring 40 as a counter contact in order to connect the electrode 19 to the high-voltage stage 15 for generating the dielectric barrier plasma when the treatment head 12 is in the inserted state. A clamping section of the clamp spring 40 surrounds the lateral surface of the connecting bolt 20, thereby establishing an electrical connection to the high-stage voltage, even during a rotational movement.

As already depicted in other figures, a snap-in connection can also be provided here to hold the electrode arrangement securely in the housing of the plasma treatment device. It is fully encompassed by the idea of the invention and conceivable in any embodiment that, in addition to a form-fit detachable connection (snap-in connection), magnetic connections and/or force-fit connections for holding the electrode arrangement in the inserted position can also be realized. It is therefore conceivable for magnetic elements to be provided in the recess 39 in the carrier 17 as well as on the drive shaft 38, said elements creating a magnetic connection when the treatment head 12 is in the inserted position.

FIGS. 6, 7 and 8 depict an embodiment example in which, alternatively or in addition to the position switches 22a, 22b, continuity testers 33a, 33b are provided as position sensors 22 (from FIGS. 1 and 2), which contact an electrically conductive ring 34 when the electrode arrangement is moved into the inserted position. The continuity testers 33a, 33b can then detect that they are in electrical contact with the electrically conductive ring 34 of the treatment head 12, whereby the correct fit of the electrode arrangement or treatment head 12 in the inserted position is assumed. Said position sensors 22 can be connected to an electrical line of the control device 14 in order to set the device to an activated operating state or a deactivated operating state.

FIGS. 7 and 8 provide two embodiments which can be used, even without continuity testers to ensure the correct fit of the electrode arrangement or treatment head 12 in the inserted position. A treatment head 12 is depicted on the left-hand side which, as previously described, comprises a carrier 17 and a dielectric 18 arranged on it.

The right-hand illustration in FIGS. 7 and 8 shows a dielectric 18 that does not require a carrier 17 and in which the dielectric 18 is thickened in width. The connecting bolt 20 protruding from the dielectric 18 is surrounded by a separate electrically insulating cover 21, said electrically insulating cover 21 protruding into the dielectric 18.

The electrically conductive ring 34 is located at the rear of the carrier 17 (left-hand side) or the rear of the dielectric 18 (right-hand side), said ring being brought into contact with the respective continuity tester 33a, 33b in the inserted position.

The dielectric 18, which is made of an electrically insulating, gel-like material, can be designed as a single piece and integrally embed the electrode. However, it is also conceivable that the dielectric 18 is formed of two or more connected layers, wherein the skin-side layer (forming the treatment surface) is soft and elastic.

FIG. 9 depicts an embodiment in which the treatment surface 18a of the dielectric comprises spacers 41, between which one or multiple gas spaces 42 are formed in which the generated plasma can spread. The spacers in FIG. 9 are nubs formed in the dielectric 18, said nubs preferably having been formed as a single piece from the gel-like material. The spacers 41 are therefore also made of a gel-like material, preferably the same material as the dielectric 18.

FIG. 10 shows an embodiment in which the electrode or treatment head is arranged in the housing 11 such that it can be moved in a translational manner. To this end, the plasma treatment device has a motor (not shown here as it is concealed), the drive shaft 38 of which comprises an eccentric bolt 38a on the end face, which engages in a form-fit manner in a slotted hole 43 of a sleeve 44 overlapping the connecting bolt 20. The sleeve 44 is connected to the dielectric 18, in the embodiment example via the carrier 17, and can be moved in a translational manner (axially in relation to the connecting bolt 20) in the housing, so that the rotation of the drive shaft 38 of the motor due to the eccentric bolt 38a, which interacts with the slotted hole 43 of the sleeve 44, converts the rotational movement of the motor into a translational to-and-fro movement (stroke movement) of the sleeve 44—and thus of the electrode arrangement. The translational movement, indicated by the arrow to the left of the treatment head 12, periodically changes its translational direction of movement—correlating with the rotational movement of the motor's drive shaft 38—whereby the translational movement can also be perceived as a vibration depending on the rotational speed of the motor.

In this case, the sleeve 44 is designed in such a way that, in the embodiment example in FIG. 10, it forms a mount inside into which the electrode arrangement or treatment head 12 can be inserted. The connection means previously described, in particular the detachable connection means such as a snap-in connection, can be utilized here to enable the correct insertion of the treatment head or the electrode arrangement on the plasma treatment device. However, it is also conceivable that the electrode arrangement (especially the dielectric with the connecting bolt) is firmly connected to the sleeve 44 and cannot be detached.

In relation to the sleeve 44, the treatment head 12 or electrode arrangement is thus fixed such that the translational movement of the sleeve 44 can be transmitted to the electrode arrangement and possibly to the treatment head 12.

In the embodiment example in FIG. 10, the connecting bolt 20 is mounted in the inner cavity of the sleeve 44 in the inserted position, wherein the connecting bolt 20 is contacted in its upper area with the counter contact 40 provided in the housing for the purpose of electrical connection to the high-voltage stage. In the area of the connecting bolt 20, the carrier 17 also protrudes with a certain section into the inner cavity of the sleeve 44 and forms a form fit and/or force fit with the inner wall of the sleeve 44. The counter contact formed by the clamp spring 40 may be firmly connected to the sleeve 44.

In particular, the translational movement, with the periodically changing movement direction, is axial to the connecting bolt 20.

### REFERENCE LIST

10 plasma treatment device
11 housing
11a upper housing part
11b lower housing part
12 treatment head
13 actuation head
14 control device
15 high-voltage stage
16 internal electrical energy source
17 carrier
17a dielectric support
18 dielectric
18a treatment surface
19 electrode
20 connecting bolt
21 electrically insulating cover
22 position sensor
22a first position switch
22 second position switch
23 line
24 guide channel
25 contact surface
26 spring contact as a counter contact
27 circuit board
28 tappet
29 snap-in section
30 motor
31 drive train
32 external energy source
33 continuity tester
34 electrically conductive ring
35 connection arrangement
36 gearwheels
37a first gearwheel arrangement
37b second gearwheel arrangement
38 drive shaft
38a eccentric bolt of the drive shaft
39 recess
40 clamp spring as a counter contact
41 spacer
42 gas space
43 slotted hole
44 sleeve

1. A plasma treatment device for treating a skin surface containing living cells with a dielectric barrier plasma, comprising:
a housing with a grip;
a treatment head arranged on the housing;
an electrode arrangement provided on the treatment head, wherein the electrode arrangement comprises at least one electrode and a dielectric that completely covers the at least one electrode with respect to a skin surface to be treated, wherein said dielectric comprises a treatment surface, wherein said dielectric is configured with the at least one electrode such that the at least one electrode is engageable with the skin surface to be treated as a counter electrode to generate the dielectric barrier plasma, and wherein the electrode arrangement is arranged in the housing such that is moveable;
a drive arrangement with at least one motor and at least one drive train positioned in the housing, wherein the at least one drive train is operatively connected with the electrode arrangement for motor driven movement;
and
a high-voltage stage arranged in the housing for generating high-voltage signals for generating the dielectric barrier plasma, wherein said high-voltage stage is in electrical contact with or is configured to be brought into electrical contact with the at least one electrode of the electrode arrangement by a connection arrangement comprising at least one high-voltage supply line,
wherein the at least one electrode is in electrical contact with or is configured to be brought into electrical contact with the high-voltage stage via the connection arrangement for generating the dielectric barrier plasma between the electrode and the skin surface surface to be treated when the skin surface to be treated is acting as the counter electrode.

2. The plasma treatment device according to claim 1, wherein the electrode arrangement is detachably arranged or insertable on the housing, and wherein the connection arrangement connects the at least one electrode to the high-voltage stage when the electrode arrangement is movably mounted on the housing in an inserted position.

3. The plasma treatment device according to claim 1, wherein the electrode arrangement is detachably arranged or insertable on the housing by snap-on connection.

4. The plasma treatment device according to claim 1 further comprising at least one position sensor designed to detect an inserted position of the electrode arrangement in the housing, wherein the high-voltage stage is switched off unless the inserted position is detected by the at least one position sensor.

5. The plasma treatment device according to claim 1 wherein the at least one electrode is led out of the dielectric with an electrical connecting bolt on a rear side of the electrode arrangement opposite the treatment surface, wherein the electrical connecting bolt has a contact surface on an end section opposite the at least one electrode which engages with a counter contact that is in the housing, wherein the counter contact and is electrically connected to the high-voltage stage for electrical contacting when the electrode arrangement is movably mounted on the housing in the inserted position.

6. The plasma treatment device according to claim 5 wherein the electrical connecting bolt forms a form fit and/or force fit with at least one part of the at least one drive train that acts about the longitudinal axis of the electrical connecting bolt when the electrode arrangement is moveably mounted on the housing in the inserted position.

7. The plasma treatment device according to claim 1 wherein the electrode arrangement in the treatment head comprises a flat carrier to which the dielectric is fixed to a dielectric support and from which an electrical connecting bolt is led out on a rear side of the flat carrier opposite the dielectric.

8. The plasma treatment device according to claim 7 wherein the flat carrier of the treatment head comprises a recess around the electrical connecting bolt on a rear side, and wherein the flat carrier is connected in a torque-proof manner to at least one part of the at least one drive train when the electrode arrangement is mounted on the housing in the inserted position.

9. The plasma treatment device according to claim 7 wherein the electrical connecting bolt is at least partially surrounded by an electrically insulating cover.

10. The plasma treatment device according to claim 1 wherein one of the preceding claims, the dielectric of the electrode arrangement is formed at least partially from a gel-like material.

11. The plasma treatment device according to claim 1 wherein the treatment surface of the dielectric comprises a spacer with elevations and with depressions between the elevations forming a gas space.

12. The plasma treatment device according to claim 10 further comprising an electric energy store in the housing that is configured to supply the high-voltage stage with electrical energy.

13. The plasma treatment device according to claim 1 wherein the high-voltage stage is connected to or configured to be connected to an external energy source.

14. The plasma treatment device according to claim 1 wherein the electrode arrangement is mounted in the housing in either a rotating manner, a rotationally oscillating manner, in a manner that allows for translational movement, and/or a manner that allows for vibrational movement.

15. The plasma treatment device of claim 10 wherein the gel-like material is a polyurethane gel.

16. The plasma treatment device of claim 15 wherein the polyurethane gel is produced from the reaction of at least one polyol with at least one suitable isocyanate, and wherein a disperse phase of the gel mobilized in the reaction is formed by the at least one polyol.

17. The plasma treatment device of claim 1, wherein the treatment surface of the dielectric has a spacer with elevations and depressions between them for forming a gas space.
